(19) United States
(12) Patent Application Publication
(10) Pub. No.: US 2011/0016542 A1
(43) Pub. Date: Jan. 20, 2011

(54) CANINE GENOME EDITING WITH ZINC FINGER NUCLEASES

(75) Inventors: Joseph Bedell, St. Louis, MO (US); Brian Buntaine, St. Louis, MO (US); Xiaoxia Cui, St. Louis, MO (US)

Correspondence Address: POLSINELLI SHUGHART PC, 700 W. 47TH STREET, SUITE 1000, KANSAS CITY, MO, 64112-1802, US

(73) Assignee: SIGMA-ALDRICH CO., St. Louis, MO (US)

(21) Appl. No.: 12/842,839

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. G01N 33/00; A01K 67/027; C12N 5/071

(52) U.S. Cl. 800/3; 800/14; 435/350

(57) ABSTRACT

The present invention provides a genetically modified canine or cell comprising at least one edited chromosomal sequence. In particular, the chromosomal sequence is edited using a zinc finger nuclease-mediated editing process. The disclosure also provides zinc finger nucleases that target specific chromosomal sequences in the canine genome.

### CROSS-REFERENCE TO RELATED APPLICATIONS

This application claims the priority of U.S. provisional application No. 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, U.S. provisional application No. 61/263,904, filed Nov. 24, 2009, U.S. provisional application No. 61/263,696, filed Nov. 23, 2009, U.S. provisional application No. 61/245,877, filed Sept. 25, 2009, U.S. provisional application No. 61/232,620, filed Aug. 10, 2009, U.S. provisional application No. 61/228,419, filed Jul. 24, 2009, and is a continuation in part of U.S. non-provisional application Ser. No. 12/592,852, filed Dec. 3, 2009, which claims priority to U.S. provisional 61/200,985, filed December 4, 2008 and US provisional application 61/205,970, filed Jan. 26, 2009, all of which are hereby incorporated by reference in their entirety.

### FIELD OF THE INVENTION

The invention generally relates to genetically modified canines or canine cells comprising at least one edited chromosomal sequence. In particular, the invention relates to the use of targeted zinc finger nucleases to edit chromosomal sequences in the canine.

### BACKGROUND OF THE INVENTION

The dog has emerged as a premier species for the study of morphology, behavior and disease. There are over 300 purebred dog populations identified worldwide, with nearly 170 recognized in the United States by the American Kennel Club (AKC). Gene flow between breeds is restricted by the pedigree barrier—registering a dog as a member of a particular breed requires that both of the dog's parents be registered members of that same breed. Most modern dog breeds are relatively young, and many of these were derived from a small number of founders, some of which is as few as six. Therefore, the physical or behavioral traits breeders wished to feature in a given breed are best represented and kept. Some physical features make certain breeds particularly successful in the show ring and hunting or performance events, and as a result, they may produce >100 litters in their lifetime. For many breeds, therefore, the purebred dogs of today represent a limited genetic pool, with disease predispositions that derive from one or a small number of recent genetic founders.

Because of the domestication history of dogs, Canine inherited disease is a challenge to breeders and dog owners. Over 360 genetic disorders in dogs have been described to date. The high susceptibility to specific diseases in particular breeds, strongly suggests that some breeds are enriched for the presence of risk alleles. 46% of genetic diseases reported in dogs are believed to occur predominantly or exclusively in one or a few breeds. Therefore, in any given breed it is likely that there is a small number of (or even one) disease alleles of strong effect and such alleles can be readily identified in samples of modest size. At least half of all described canine diseases resemble specific human disorders, which include but not limited to cancer, deafness, heart disease, cataracts, hip dysplasia, thyroid disease, bloat, autoimmune diseases, progressive retinal atrophy, and epilepsy.

The progress of ongoing research into the causes and treatments of these canine and human diseases is hampered by the onerous task of developing an animal model which incorporates the genes proposed to be involved in the development or severity of the diseases. Conventional methods such as gene knockout technology may be used to edit a particular gene in a potential model organism in order to develop an animal model of a certain human or canine disease. However, gene knockout technology may require months or years to construct and validate the proper knockout models. In addition, genetic editing via gene knockout technology has been reliably developed in only a limited number of organisms such as mice. Although rodent systems are genetically tractable, but the mutations typically represent induced rather than naturally arising alleles, because it often requires specific carcinogenic exposures. Therefore, the results are often of limited direct relevance to human disease because of profound differences in physiology. In addition, even in a best case scenario, mice typically show low intelligence, making mice a poor choice of organism in which to study complex disorders.

Unlike most rodent models, cancer in dogs is a naturally occurring disease, with both inherited and sporadic forms noted for all common types of cancer. In comparison, in dog system, the physiology, disease presentation, histopathological appearance, biological behavior, and response to clinical therapy often mimic human diseases closely. Also importantly, the structure of dog breeds has the potential to dramatically reduce the problems associated with heterogeneity and genetic complexity of common disease inheritance and makes dog disease genes good targets for various research purposes. The availability of a high-quality draft sequence makes the dog system even more attractive for research in dog traits, dog inherited diseases and human common diseases. Therefore various dog model organisms are needed for canine and human diseases research and therapy development.

Other than for breeding purposes and disease research, dogs as pets to humans, excessive barking, hair shedding, allergen, sterility are targets for desirable traits development. Canis familiaris allergen 1 (Can f 1) is a lipocalin allergen produced by dogs, which is found in the dog's saliva and dander. Excessive barking may also contribute to higher levels of allergen dispersal. Hypoallergenic dogs with reduced levels of Can f 1 are desirable. Nonallergenic dogs with no Can f 1 would be even more desirable.

Therefore, a need exists for animals with modification to one or more genes associated with various canine or human diseases and desirable pet traits to be used as model organisms in which to study these diseases and traits. The genetic modifications may include gene knockouts, expression, modified expression, or over-expression of alleles that either cause or are associated with human or canine diseases and desirable traits. Further, a need exists for modification of one or more genes associated with human or canine diseases and desirable traits in a variety of organisms in order to develop appropriate animal models of diseases such as cancer, deafness, heart disease, blindness, and epilepsy; and animal models of traits such as non-excessive-barking, non-hair-shedding and hypoallergenic.

### SUMMARY OF THE INVENTION

One aspect of the present disclosure encompasses a genetically modified canine comprising at least one edited chromosomal sequence.

A further aspect provides a canine embryo comprising at least one RNA molecule encoding a zinc finger nuclease that recognizes a chromosomal sequence and is able to cleave a site in the chromosomal sequence, and, optionally, (i) at least one donor polynucleotide comprising a sequence that is flanked by an upstream sequence and a downstream sequence, the upstream and downstream sequences having substantial sequence identity with either side of the site of cleavage or (ii) at least one exchange polynucleotide comprising a sequence that is substantially identical to a portion of the chromosomal sequence at the site of cleavage and which further comprises at least one nucleotide change.

Another aspect provides a genetically modified canine cell comprising at least one edited chromosomal sequence.

Yet another aspect encompasses a method for assessing the effect of an agent in an animal. The method comprises contacting a genetically modified animal comprising at least one edited chromosomal sequence encoding a canine or human disease-related protein with the agent, and comparing results of a selected parameter to results obtained from contacting a wild-type animal with the same agent. The selected parameter is chosen from (a) rate of elimination of the agent or its metabolite(s); (b) circulatory levels of the agent or its metabolite(s); (c) bioavailability of the agent or its metabolite(s); (d) rate of metabolism of the agent or its metabolite(s); (e) rate of clearance of the agent or its metabolite(s); (f) toxicity of the agent or its metabolite(s); and (g) efficacy of the agent or its metabolite(s).

Still yet another aspect encompasses a method for assessing the therapeutic potential of an agent in an animal. The method includes contacting a genetically modified animal comprising at least one edited chromosomal sequence encoding a canine or human disease-related protein, and comparing the results of a selected parameter to results obtained from a wild-type animal with contact with an agent. The selected parameter may be chosen from a) spontaneous behaviors; b) performance during behavioral testing; c) physiological anomalies; d) abnormalities in tissues or cells; e) biochemical function; and f) molecular structures.

Other aspects and features of the disclosure are described more thoroughly below.

### DETAILED DESCRIPTION OF THE INVENTION

The present disclosure provides a genetically modified animal or animal cell comprising at least one edited chromosomal sequence encoding a protein associated with canine- or human-related diseases or canine traits. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock out” or a “conditional knock out.” Similarly, a genetically modified animal comprising an integrated sequence may be termed a “knock in” or a “conditional knock in.” As detailed below, a knock in animal may be a humanized animal. Furthermore, a genetically modified animal comprising a modified chromosomal sequence may comprise a targeted point mutation(s) or other modification such that an altered protein product is produced. The chromosomal sequence encoding the protein associated with canine- or human-related diseases or canine traits generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with canine- or human-related diseases or canine traits using targeted zinc finger nuclease technology is rapid, precise, and highly efficient.

### (I) Genetically Modified Canine

One aspect of the present disclosure provides a genetically modified canine in which at least one chromosomal sequence encoding a disease or trait related protein has been edited. For example, the edited chromosomal sequence may be inactivated such that the sequence is not transcribed and/or a functional disease or trait-related protein is not produced. Alternatively, the edited chromosomal sequence may be modified such that it codes for an altered disease or trait-related protein. For example, the chromosomal sequence may be modified such that at least one nucleotide is changed and the expressed disease or trait -related protein comprises at least one changed amino acid residue (missense mutation). The chromosomal sequence may be modified to comprise more than one missense mutation such that more than one amino acid is changed. Additionally, the chromosomal sequence may be modified to have a three nucleotide deletion or insertion such that the expressed disease or trait-related protein comprises a single amino acid deletion or insertion, provided such a protein is functional. For example, a protein coding sequence may be inactivated such that the protein is not produced. Alternatively, a microRNA coding sequence may be inactivated such that the microRNA is not produced. Furthermore, a control sequence may be inactivated such that it no longer functions as a control sequence. The modified protein may have altered substrate specificity, altered enzyme activity, altered kinetic rates, and so forth. Furthermore, the edited chromosomal sequence may comprise an integrated sequence and/or a sequence encoding an orthologous protein associated with a disease or a trait. The genetically modified canine disclosed herein may be heterozygous for the edited chromosomal sequence encoding a protein associated with a disease or a trait. Alternatively, the genetically modified canine may be homozygous for the edited chromosomal sequence encoding a protein associated with a disease or a trait.

In one embodiment, the genetically modified canine may comprise at least one inactivated chromosomal sequence encoding a disease or trait -related protein. The inactivated chromosomal sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional disease or trait-related protein is not produced. The inactivated chromosomal sequence comprises no exogenously introduced sequence. Such a canine may be termed a “knockout.” Also included herein are genetically modified canines in which two, three, four, five, six, seven, eight, nine, or ten or more chromosomal sequences encoding proteins associated with a disease or a trait.

In another embodiment, the genetically modified canine may comprise at least one edited chromosomal sequence encoding an orthologous protein associated with a disease or a trait. The edited chromosomal sequence encoding an orthologous disease or trait-related protein may be modified such that it codes for an altered protein. For example, the edited chromosomal sequence encoding a disease or trait-related protein may comprise at least one modification such that an altered version of the protein is produced. In some embodiments, the edited chromosomal sequence comprises at least one modification such that the altered version of the disease or trait- related protein results in the disease or the trait in the animal. In other embodiments, the edited chromosomal sequence encoding a disease or trait-related protein comprises at least one modification such that the altered version of the protein protects against a disease or does not form a trait in the animal. The modification may be a missense mutation in which substitution of one nucleotide for another nucleotide changes the identity of the coded amino acid.

In yet another embodiment, the genetically modified canine may comprise at least one chromosomally integrated sequence. The chromosomally integrated sequence may encode an orthologous disease or trait-related protein, an endogenous disease or trait-related protein, or combinations of both. For example, a sequence encoding an orthologous protein or an endogenous protein may be integrated into a chromosomal sequence encoding a protein such that the chromosomal sequence is inactivated, but wherein the exogenous sequence may be expressed. In such a case, the sequence encoding the orthologous protein or endogenous protein may be operably linked to a promoter control sequence. Alternatively, a sequence encoding an orthologous protein or an endogenous protein may be integrated into a chromosomal sequence without affecting expression of a chromosomal sequence. For example, a sequence encoding a canine or human disease-related protein may be integrated into a “safe harbor” locus, such as the Rosa26 locus, HPRT locus, or AAV locus. In one iteration of the disclosure an animal comprising a chromosomally integrated sequence encoding disease- or trait-related protein may be called a “knock-in”, and it should be understood that in such an iteration of the animal, no selectable marker is present. An animal comprising a chromosomally integrated sequence encoding a canine or human disease-related protein may be called a “knock-in.” The present disclosure also encompasses genetically modified animals in which two, three, four, five, six, seven, eight, nine, or ten or more sequences encoding protein(s) associated with a disease or a trait are integrated into the genome.

In an exemplary embodiment, the genetically modified canine may be a “humanized” canine comprising at least one chromosomally integrated sequence encoding a functional human disease or trait-related protein. The functional human disease or trait-related protein may have no corresponding ortholog in the genetically modified canine. Alternatively, the wild-type canine from which the genetically modified canine is derived may comprise an ortholog corresponding to the functional human disease or trait-related protein. In this case, the orthologous sequence in the “humanized” canine is inactivated such that no functional protein is made and the “humanized” canine comprises at least one chromosomally integrated sequence encoding the human disease or trait-related protein. Those of skill in the art appreciate that “humanized” canines may be generated by crossing a knock out canine with a knock in canine comprising the chromosomally integrated sequence.

The chromosomally integrated sequence encoding a disease or trait-related protein may encode the wild type form of the protein. Alternatively, the chromosomally integrated sequence encoding a disease or trait-related protein may comprise at least one modification such that an altered version of the protein is produced. In some embodiments, the chromosomally integrated sequence encoding a disease or trait-related protein comprises at least one modification such that the altered version of the protein produced causes a disease or forms a trait. In other embodiments, the chromosomally integrated sequence encoding a disease or trait-related protein comprises at least one modification such that the altered version of the protein protects against the development of a disease or an undesirable trait.

In yet another embodiment, the genetically modified canine may comprise at least one edited chromosomal sequence encoding a disease or trait-related protein such that the expression pattern of the protein is altered. For example, regulatory regions controlling the expression of the protein, such as a promoter or transcription binding site, may be altered such that the disease or trait-related protein is over-produced, or the tissue-specific or temporal expression of the protein is altered, or a combination thereof. Alternatively, the expression pattern of the disease or trait-related protein may be altered using a conditional knockout system. A non-limiting example of a conditional knockout system includes a Cre-lox recombination system. A Cre-lox recombination system comprises a Cre recombinase enzyme, a site-specific DNA recombinase that can catalyse the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence, such as a chromosomal sequence encoding a disease or trait-related protein. The genetically modified canine comprising the lox-flanked chromosomal sequence encoding a disease or trait-related protein may then be crossed with another genetically modified canine expressing Cre recombinase. Progeny comprising the lox-flanked chromosomal sequence and the Cre recombinase are then produced, and the lox-flanked chromosomal sequence encoding a disease or trait-related protein is recombined, leading to deletion or inversion of the chromosomal sequence encoding the protein. Expression of Cre recombinase may be temporally and conditionally regulated to effect temporally and conditionally regulated recombination of the chromosomal sequence encoding a disease or trait-related protein.

Exemplary examples of canine chromosomal sequences to be edited include those that code for proteins relating to dog allergy (Can f 1), disproportionately short limbs (fibroblast growth factor-4, FGF4), small size (insulin like growth factor-1, IGF-1), fur smooth versus wire texture (T-spondin-2, PSPO2 for wire hair), long versus short fur (fibroblast growth factor-5, FGF5), curly versus straight fur (keratin71, KRT71), hairless (fork head box transcription factor family, FOX13), coat color (melanocortin 1 receptor, Mclr; Agouti; and β-defensin, CBD103), complete or partial absence of pigmentation (microphthalmia-associated transcription factor, MITF). A dog with Can f 1 “knock-out” or modification may be hypoallergic, or non-allergic, and/or without excessive barking. Those of skill in the art appreciate that other proteins are involved in coat color, coat patter, and hair length, but the genetic loci have not been determined.

Exemplary examples of canine chromosomal sequences to be edited include those that code for proteins relating to canine and human diseases. Non-limiting example include vision disorders, kidney cancer, narcolepsy, rheumatoid arthritis, SCID, keratin-associated diseases, cystinuria, bleeding disorders, ceroid lipofuscinosis and copper toxicosis. In one embodiment, the genetically modified canine may comprise an edited chromosomal sequence encoding hypocretin-2-receptor gene HCRTR2. A mutation in the hypocretin-2-receptor gene HCRTR2 causes canine narcolepsy. Although narcolepsy is a relatively rare disease in humans, sleep disorders in general are common in the human population. Clinical studies established that hypocretin deficiency is associated with most cases of narcolepsy in humans. In addition, Hypocretin has a key role in circadian clock-dependent alertness as well as integrating hypothalamic signals for neuroendocrine release and for regulating metabolic rate, appetite, mood and sleep. Therefore, the hypocretin system might be a therapeutic target not only in the treatment of narcolepsy, but also for more common sleep disturbances. A genetically modified dog comprising modified HCRTR2 can be used as a model organism for understanding human sleep disorder and the therapy thereof.

Canine hereditary multifocal renal cystadenocarcinoma and nodular dermatofibrosis (RCND) is a naturally occurring inherited cancer syndrome in German shepherd dogs and is characterized by multifocal tumors in kidneys and skin. RCND locus overlaps with human BHD (Birt-Hogg-Dubé) disease locus. BHD is a multisystem disorder in humans that bears strong similarity to RCND, indicating that the same gene might be responsible for both the human and canine disease. A single base change leads to alteration of a highly conserved amino acid, resulting in a disease-associated mutation in the canine-encoded protein folliculin. A dog comprising genetically edited RCND can be used as an animal model for a rare and intriguing human disorder.

In one embodiment, the genetically modified canine may comprise an edited chromosomal sequence encoding protein folliculin, wherein the edited chromosomal sequence comprises a mutation such that an altered folliculin is produced. The mutation may also be a nonsense mutation in which substitution of one nucleotide for another introduces a stop codon, a deletion mutation in which one or more nucleotides are deleted from the chromosomal sequence, or an insertion mutation in which one or more nucleotides are introduced into the chromosomal sequence. Accordingly, the nonsense, deletion, or insertion mutation “inactivates” the sequence such that folliculin protein is not produced. Thus, a genetically modified canine comprising an inactivated folliculin chromosomal sequence may be used as a model organism for human kidney cancer research.

Retinitis pigmentosa (RP) is a human retinal degeneration that results in blindness, affecting approximately 1 in 4,000 people. At least 37 RP loci in the human genome have been identified. Progressive retinal atrophie (PRA) is a form of Canine RP. Mutations in the canine RPE65 gene cause near-total blindness in infancy. The RPE65−/−dog, suffers from early and severe visual impairment that is similar to that seen in humans with such a disease. Genetically modified dogs comprising RPE65 edited sequence can be used as a model organism providing a research system for cell biology and pathogenesis of these diseases and for therapeutic interventions.

In another embodiment, the genetically modified canine may comprise an edited chromosomal sequence encoding RPE65 protein, wherein the edited chromosomal sequence comprises at least one modification such that an altered version of RPE65 protein is produced. The modification may be a missense mutation in which substitution of one nucleotide for another nucleotide changes the identity of the coded amino acid. The RPE65 coding region may be edited to comprise more than one missense mutation such that more than one amino acid is changed. Additionally, the chromosomal region may be modified to have a three nucleotide deletion or insertion such that the expressed RPE65 protein comprises a single amino acid deletion or insertion, provided such a protein is functional. Those of skill in the art will appreciate that many different modifications are possible in the RPE65 coding region. The modified RPE65 coding region may give rise to a RPE65 protein associated with restorable PRA. In one embodiment, the genetically modified canine comprising a modified RPE65 chromosomal region may have less susceptibility to visual impairment. In other embodiments, the genetically modified canine comprising a modified RPE65 chromosomal region may have a later on-set visional impairment than a canine in which the RPE65 chromosomal region is not modified.

In still another embodiment, the genetically modified canine may comprise an edited chromosomal sequence encoding HCRTR2, RCND, RPE65, or combinations thereof. The edited chromosomal sequence may comprise at least one modification such that an altered version of HCRTR2, RCND, or RPE65 is produced. The chromosomal sequence may be modified to contain at least one nucleotide change such at the expressed protein comprises at least one amino acid change as detailed above. Alternatively, the edited chromosomal sequence may comprise a mutation such that the sequence is inactivated and no protein is made or a defective protein is made. As detailed above, the mutation may comprise a deletion, an insertion, or a point mutation. The genetically modified canine comprising an edited HCRTR2, RCND, and/or RPE65 chromosomal sequence may have a different susceptibility to narcolepsy, kidney cancer and/or vision impairment than a canine in which said chromosomal region(s) is not edited.

The present disclosure also encompasses a genetically modified canine comprising any combination of the above described chromosomal alterations. For example, the genetically modified canine may comprise an inactivated Can f 1 and/or Agouti chromosomal sequence, a modified FGF4 chromosomal sequence, and/or a modified or inactivated HCRTR2, RCND, and/or RPE65 chromosomal sequence.

Additionally, the human or canine disease- or canine trait-related gene may be modified to include a tag or reporter gene, as is well-known. Reporter genes include those encoding selectable markers such as cloramphenicol acetyltransferase (CAT) and neomycin phosphotransferase (neo), and those encoding a fluorescent protein such as green fuorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the targeted gene to create a gene fusion. A reporter sequence can be integrated in a targeted manner in the targeted gene, for example the reporter sequences may be integrated specifically at the 5′ or 3′ end of the targeted gene. The two genes are thus under the control of the same promoter elements and are transcribed into a single messenger RNA molecule. Alternatively, the reporter gene may be used to monitor the activity of a promoter in a genetic construct, for example by placing the reporter sequence downstream of the target promoter such that expression of the reporter gene is under the control of the target promoter, and activity of the reporter gene can be directly and quantitatively measured, typically in comparison to activity observed under a strong consensus promoter. It will be understood that doing so may or may not lead to destruction of the targeted gene.

The genetically modified canine may be heterozygous for the edited chromosomal sequence or sequences. In other embodiments, the genetically modified canine may be homozygous for the edited chromosomal sequence or sequences. In each of the foregoing iterations of suitable canines for the invention, the canine does not include exogenously introduced, randomly integrated transposon sequences

The genetically modified canine may be a member of one of the following breeds: Labrador retriever, Golden retriever, Beagle, German shepherd, Dachshund, Yorkshire terrier, Boxer, Poodle, Shih tzu, Chihuahua, Miniature schnauzer, Pug dog, Pomeranian, Cocker spaniel, Rottweiler, Bulldog, Shetland sheepdog, Boston terrier, Miniature pinscher, Maltese, German shorthaired pointer, Doberman pinscher, Siberian husky, Pembroke welsh corgi, Basset hound, Bichon frise, and other existing or non-existing breeds. As used herein, the term “canine” encompasses embryos, fetuses, newborn puppies, juveniles, and adult canine organisms.

### (II) Genetically Modified Canine Cells

A further aspect of the present disclosure provides genetically modified canine cells or cell lines comprising at least one edited chromosomal sequence. The disclosure also encompasses a lysate of said cells or cell lines. The genetically modified canine cell (or cell line) may be derived from any of the genetically modified canines disclosed herein. Alternatively, the chromosomal sequence may be edited in a canine cell as detailed below.

The canine cell may be any established cell line or a primary cell line that is not yet described. The cell line may be adherent or non-adherent, or the cell line may be grown under conditions that encourage adherent, non-adherent or organotypic growth using standard techniques known to individuals skilled in the art. The canine cell or cell line may be derived from lung (e.g., AKD cell line), kidney (e.g., CRFK cell line), liver, thyroid, fibroblasts, epithelial cells, myoblasts, lymphoblasts, macrophages, tumor cells, and so forth. Additionally, the canine cell or cell line may be a canine stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

Similar to the genetically modified canines, the genetically modified canine cells may be heterozygous or homozygous for the edited chromosomal sequence or sequences.

### (III) Zinc Finger-Mediated Genome Editing

In general, the genetically modified canine or canine cell, as detailed above in sections (I) and (II), respectively, is generated using a zinc finger nuclease-mediated genomic editing process. The process for editing a canine chromosomal sequence comprises: (a) introducing into a canine embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration, the sequence flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence. The embryo used in the above described method typically is a fertilized one-cell stage embryo.

Components of the zinc finger nuclease-mediated method of genome editing are described in more detail below.

### (a) Zinc Finger Nuclease

The method comprises, in part, introducing into a canine embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

### (i) Zinc Finger Binding Domain

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publically available web-based tools for identifying potential target sites in DNA sequences and designing zinc finger binding domains may be found at http://www.zincfingertools.org and http://bindr.gdcb.iastate.edu/ZiFiT/, respectively (Mandell et al. (2006) Nuc. Acid Res. 34:W516-W523; Sander et al. (2007) Nuc. Acid Res. 35:W599-W605).

A zinc finger DNA binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

### (ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a Fok I cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from I to K in one cleavage monomer to produce an engineered cleavage monomer designated “E490K:1538K” and by mutating positions 486 from Q to E and 499 from I to L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

### (b) Optional Exchange Polynucleotide

The method for editing chromosomal sequences may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may be at least about 80, 81, 82, 83, 84, 85, 86, 87, 88, 89, 90, 91, 92, 93, 94, 95, 96, 97, 98, or 99% identical a region of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 by to about 10,000 by in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 by in length. In other embodiments, the sequence in the exchange polynucleotide may be about 5500, 6000, 6500, 6000, 6500, 7000, 7500, 8000, 8500, 9000, 9500, or 10,000 by in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (c) Optional Donor Polynucleotide

The method for editing chromosomal sequences may further comprise introducing at least one donor polynucleotide comprising a sequence for integration into the embryo or cell. A donor polynucleotide comprises at least three components: the sequence to be integrated that is flanked by an upstream sequence and a downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide may be a DNA plasmid.

The donor polynucleotide comprises a sequence for integration. The sequence for integration may be a sequence endogenous to the canine or it may be an exogenous sequence. Additionally, the sequence to be integrated may be operably linked to an appropriate control sequence or sequences. The size of the sequence to be integrated can and will vary. In general, the sequence to be integrated may range from about one nucleotide to several million nucleotides.

The donor polynucleotide also comprises upstream and downstream sequence flanking the sequence to be integrated. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 by to about 2500 bp. In one embodiment, an upstream or downstream sequence may comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 by to about 2000 bp, about 600 bp to about 1000 bp, or more particularly about 700 bp to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for editing a chromosomal sequence by integrating a sequence, the double stranded break introduced into the chromosomal sequence by the zinc finger nuclease is repaired, via homologous recombination with the donor polynucleotide, such that the sequence is integrated into the chromosome. The presence of a double-stranded break facilitates integration of the sequence. A donor polynucleotide may be physically integrated or, alternatively, the donor polynucleotide may be used as a template for repair of the break, resulting in the introduction of the sequence as well as all or part of the upstream and downstream sequences of the donor polynucleotide into the chromosome. Thus, the endogenous chromosomal sequence may be converted to the sequence of the donor polynucleotide.

### (d) Delivery of Nucleic Acids

To mediate zinc finger nuclease genome editing, at least one nucleic acid molecule encoding a zinc finger nuclease and, optionally, at least one exchange polynucleotide or at least one donor polynucleotide is delivered into the canine embryo or cell. Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and an exchange (or donor) polynucleotide are introduced into an embryo or cell, the ratio of exchange (or donor) polynucleotide to nucleic acid encoding a zinc finger nuclease may range from about 1:10 to about 10:1. In various embodiments, the ratio of exchange (or donor) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one exchange (or donor) polynucleotide is introduced into an embryo or cell, the nucleic acids may be introduced simultaneously or sequentially. For example, nucleic acids encoding the zinc finger nucleases, each specific for a distinct recognition sequence, as well as the optional exchange (or donor) polynucleotides, may be introduced at the same time. Alternatively, each nucleic acid encoding a zinc finger nuclease, as well as the optional exchange (or donor) polynucleotides, may be introduced sequentially.

### (e) Culturing the Embryo or Cell

The method for editing a chromosomal sequence using a zinc finger nuclease-mediated process further comprises culturing the embryo or cell comprising the introduced nucleic acid(s) to allow expression of the zinc finger nuclease.

An embryo may be cultured in vitro (e.g., in cell culture). Typically, the canine embryo is cultured for a short period of time at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the canine species. Routine optimization may be used, in all cases, to determine the best culture conditions for a particular species of embryo. In some cases, a cell line may be derived from an in vitro-cultured embryo (e.g., an embryonic stem cell line).

Preferably, the canine embryo will be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal generally will comprise the disrupted chromosomal sequence(s) in every cell of the body.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no exchange (or donor) polynucleotide, the zinc finger nuclease recognizes, binds, and cleaves the target sequence in the chromosomal sequence of interest. The double-stranded break introduced by the zinc finger nuclease is repaired by the error-prone non-homologous end-joining DNA repair pathway. Consequently, a deletion, insertion, or nonsense mutation may be introduced in the chromosomal sequence such that the sequence is inactivated.

In cases in which the embryo or cell comprises an expressed zinc finger nuclease as well as an exchange (or donor) polynucleotide, the zinc finger nuclease recognizes, binds, and cleaves the target sequence in the chromosome. The double-stranded break introduced by the zinc finger nuclease is repaired, via homologous recombination with the exchange (or donor) polynucleotide, such that a portion of the chromosomal sequence is converted to the sequence in the exchange polynucleotide or the sequence in the donor polynucleotide is integrated into the chromosomal sequence. As a consequence, the chromosomal sequence is modified.

The genetically modified canines disclosed herein may be crossbred to create animals comprising more than one edited chromosomal sequence or to create animals that are homozygous for one or more edited chromosomal sequences. Those of skill in the art will appreciate that many combinations are possible. Moreover, the genetically modified canines disclosed herein may be crossed with other canines to combine the edited chromosomal sequence with other genetic backgrounds. By way of non-limiting example, suitable genetic backgrounds may include wild-type, natural mutations giving rise to known canine phenotypes, targeted chromosomal integration, non-targeted integrations, etc.

(IV) Applications

The animals and cells disclosed herein may have several applications. In one embodiment, the genetically modified canine comprising at least one edited chromosomal sequence may exhibit a phenotype desired by humans. For example, inactivation of the chromosomal sequence encoding Can f 1 may result in dogs that are hypoallergenic or non-allergenic, and/or without excessive barking. In other embodiments, the canine comprising at least one edited chromosomal sequence may be used as a model to study the genetics of coat color, coat pattern, and/or hair growth, body size, leg length versus width, and skull shape. Additionally, a canine comprising at least one disrupted chromosomal sequence may be used as a model to study a disease or condition that affects humans, canines or other animals. Non-limiting examples of suitable diseases or conditions include cancer, deafness, heart disease, cataracts, hip dysplasia, thyroid disease, bloat, autoimmune diseases, progressive retinal atrophy, and epilepsy. Additionally, the disclosed canine cells and lysates of said cells may be used for similar research purposes.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor or exchange polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### EXAMPLES

The following examples are included to illustrate the invention.

### Example 1

### Genome Editing of Can f 1 Locus

Zinc finger nucleases (ZFNs) that target and cleave the Can f 1 locus of canine may be designed, assembled, and validated using strategies and procedures previously described (see Geurts et al. Science (2009) 325:433). ZFN design made use of an archive of pre-validated 1-finger and 2-finger modules. The canine Can f 1 gene region may be scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding pairs of ZFNs may be produced using known molecular biology techniques. The mRNA may be transfected into canine cells. Control cells may be injected with mRNA encoding GFP. Active ZFN pairs may be identified by detecting ZFN-induced double strand chromosomal breaks using the Cel-1 nuclease assay. This assay may detect alleles of the target locus that deviate from wild type as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture may result in mismatches forming between heteroduplexes of the WT and mutant alleles. A DNA “bubble” formed at the site of mismatch may be cleaved by the surveyor nuclease Cel-1, and the cleavage products can be resolved by gel electrophoresis. This assay may identify a pair of active ZFNs that edited the Can f 1 locus.

To mediate editing of the Can f 1 gene locus in animals, fertilized canine embryos may be microinjected with mRNA encoding the active pair of ZFNs using standard procedures (e.g., see Geurts et al. (2009) supra). The injected embryos may be either incubated in vitro, or transferred to pseudopregnant female canines to be carried to parturition. The resulting embryos/fetus, or the toe/tail clip of live born animals may be harvested for DNA extraction and analysis. DNA may be isolated using standard procedures. The targeted region of the Can f 1 locus may be PCR amplified using appropriate primers. The amplified DNA may be subcloned into a suitable vector and sequenced using standard methods.

### Example 2

### Genome Editing of HCRTR2 in a Model Organism

ZFN-mediated genome editing may be used to study the effects of a “knockout” mutation in a canine or human disease-related chromosomal sequence, such as a chromosomal sequence encoding the hypocretin receptor protein, in a genetically modified model animal and cells derived from the animal. Such a model animal may be a canine. In general, ZFNs that bind to the canine chromosomal sequence encoding the hypocretin receptor associated with canine narcolepsy may be used to introduce a deletion or insertion such that the coding region of the HCRTR2 gene is disrupted such that a functional hypocretin receptor protein may not be produced.

Suitable fertilized embryos may be microinjected with capped, polyadenylated mRNA encoding the ZFN essentially as detailed above in Example 1. The frequency of ZFN-induced double strand chromosomal breaks may be determined using the Cel-1 nuclease assay, as detailed above. The sequence of the edited chromosomal sequence may be analyzed as described above. The development of narcolepsy symptoms and disorders caused by the hypocretin receptor “knockout” may be assessed in the genetically modified canine or progeny thereof. Furthermore, molecular analyses of narcolepsy-related pathways may be performed in cells derived from the genetically modified animal comprising a HCRTR2 “knockout”.

### Example 3

### Generation of a Humanized Canine Expressing a Mutant Form of Human BHD

BHD is a multisystem disorder in humans that has strong similarity to RCND, a naturally occurring inherited canine cancer syndrome. RCND locus overlaps with human BHD locus in genome comparison. A single base change at RCND locus leads to alteration of a disease-associated protein folliculin. ZFN-mediated genome editing may be used to generate a humanized canine wherein the canine RCND locus is replaced with a mutant form of the human BHD locus comprising one or more mutations. Such a humanized canine may be used to study the development of the diseases associated with the mutant human BHD protein. In addition, the humanized canine may be used to assess the efficacy of potential therapeutic agents targeted at the pathway leading to kidney cancer comprising BHD.

The genetically modified canine may be generated using the methods described in the Examples above. However, to generate the humanized canine, the ZFN mRNA may be co-injected with the human chromosomal sequence encoding the mutant BHD protein into the canine embryo. The canine chromosomal sequence may then be replaced by the mutant human sequence by homologous recombination, and a humanized canine expressing a mutant form of the BHD protein may be produced.

What is claimed is:

1. A genetically modified canine comprising at least one edited chromosomal sequence encoding a canine or human disease-related protein.

2. The genetically modified canine of claim 1, wherein the edited chromosomal sequence is inactivated, modified, or comprises an integrated sequence.

3. The genetically modified canine of claim 1, wherein the edited chromosomal sequence is inactivated such that the canine or human disease-related protein is not produced.

4. The genetically modified canine of claim 3, further comprising at least one chromosomally integrated sequence encoding a canine or human disease-related protein.

5. The genetically modified animal of claim 1, wherein the canine or human disease is chosen from cancer, deafness, heart disease, cataracts, hip dysplasia, thyroid disease, bloat, autoimmune diseases, progressive retinal atrophy, epilepsy, and other human common diseases and canine diseases.

6. The genetically modified canine of claim 1, wherein the canine is heterozygous or homozygous for the at least one edited chromosomal sequence.

7. The genetically modified canine of claim 1, wherein the canine is an embryo, a juvenile, or an adult.

8. The genetically modified canine of claim 1, wherein the protein is a human disease-related protein.

9. A canine embryo comprising at least one RNA molecule encoding a zinc finger nuclease that recognizes a chromosomal sequence encoding a canine or human disease-related protein, and, optionally, at least one donor polynucleotide comprising a sequence encoding a canine or human disease-related protein.

10. The canine embryo of claim 9, wherein the canine or human disease-related protein is chosen from cancer, deafness, heart disease, cataracts, hip dysplasia, thyroid disease, bloat, autoimmune diseases, progressive retinal atrophy, epilepsy, and other human common diseases and canine diseases related proteins.

11. The non-human embryo of claim 9, wherein the protein is a human disease-related protein.

12. A genetically modified canine cell, the cell comprising at least one edited chromosomal sequence encoding a canine or human disease-related protein.

13. The genetically modified cell of claim 12, wherein the edited chromosomal sequence is inactivated, modified, or comprises an integrated sequence.

14. The genetically modified cell of claim 12, wherein the edited chromosomal sequence is inactivated such that the canine or human disease-related protein is not produced.

15. The genetically modified cell of claim 14, further comprising at least one chromosomally integrated sequence encoding a canine or human disease-related protein.

16. The genetically modified cell of claim 13, wherein the canine or human disease-related protein is chosen from cancer, deafness, heart disease, cataracts, hip dysplasia, thyroid disease, bloat, autoimmune diseases, progressive retinal atrophy, epilepsy, and other human common diseases and canine diseases.

17. The genetically modified cell of claim 12, wherein the cell is heterozygous or homozygous for the at least one edited chromosomal sequence.

18. The genetically modified cell of claim 12, wherein the protein is a human disease-related protein.

19. A method for assessing the effect of an agent in a canine, the method comprising contacting a genetically modified canine comprising at least one edited chromosomal sequence encoding a canine or human disease-related protein, and comparing results of a selected parameter to results obtained from contacting a wild-type canine with the same agent, wherein the selected parameter is chosen from:
a) rate of elimination of the agent or its metabolite(s);
b) circulatory levels of the agent or its metabolite(s);
c) bioavailability of the agent or its metabolite(s);
d) rate of metabolism of the agent or its metabolite(s);
e) rate of clearance of the agent or its metabolite(s);
f) toxicity of the agent or its metabolite(s); and
g) efficacy of the agent or its metabolite(s).

20. The method of claim 19, wherein the agent is a pharmaceutically active ingredient, a drug, a toxin, or a chemical.

21. The method of claim 19, wherein the at least one edited chromosomal sequence is inactivated such that the canine or human disease-related protein is not produced, and wherein the animal further comprises at least one chromosomally integrated sequence encoding a canine or human disease-related protein.

22. The method of claim 19, wherein the canine or human disease is chosen from cancer, deafness, heart disease, cataracts, hip dysplasia, thyroid disease, bloat, autoimmune diseases, progressive retinal atrophy, epilepsy, and other human common diseases and canine diseases.

23. The method of claim 19, wherein the canine is a dog of a breed chosen from Labrador retriever, Golden retriever, Beagle, German shepherd, Dachshund, Yorkshire terrier, Boxer, Poodle, Shih tzu, Chihuahua, Miniature schnauzer, Pug dog, Pomeranian, Cocker spaniel, Rottweiler, Bulldog, Shetland sheepdog, Boston terrier, Miniature pinscher, Maltese, German shorthaired pointer, Doberman pinscher, Siberian husky, Pembroke welsh corgi, Basset hound, Bichon frise, and other existing or non-existing breeds.

24. A method for assessing the therapeutic potential of an agent in a canine, the method comprising contacting a genetically modified canine comprising at least one edited chromosomal sequence encoding a canine or human disease-related protein, and comparing results of a selected parameter to results obtained from a wild-type canine with contact with the same agent, wherein the selected parameter is chosen from:
a) spontaneous behaviors;
b) performance during behavioral testing;
c) physiological anomalies;
d) abnormalities in tissues or cells;
e) biochemical function; and
f) molecular structures.

25. The method of claim 24, wherein the agent is a pharmaceutically active ingredient, a drug, a toxin, or a chemical.

26. The method of claim 24, wherein the at least one edited chromosomal sequence is inactivated such that the canine or human disease-related protein is not produced, and wherein the canine further comprises at least one chromosomally integrated sequence encoding a canine or human-related protein.

27. The method of claim 24, wherein the canine or human disease is chosen from cancer, deafness, heart disease, cataracts, hip dysplasia, thyroid disease, bloat, autoimmune diseases, progressive retinal atrophy, epilepsy, and other human common diseases and canine diseases.

28. The method of claim 24, wherein the canine is a dog of a breed chosen from Labrador retriever, Golden retriever, Beagle, German shepherd, Dachshund, Yorkshire terrier, Boxer, Poodle, Shih tzu, Chihuahua, Miniature schnauzer, Pug dog, Pomeranian, Cocker spaniel, Rottweiler, Bulldog, Shetland sheepdog, Boston terrier, Miniature pinscher, Maltese, German shorthaired pointer, Doberman pinscher, Siberian husky, Pembroke welsh corgi, Basset hound, Bichon frise, and other existing or non-existing breeds.

29. The genetically modified canine of claim 1, wherein the edited chromosomal sequence encodes a protein chosen from Can f 1, FGF4, IGF-1, PSPO2, FGF5, KRT71, FOX13, Mclr, Agouti, CBD103, MITF, and combinations thereof.

30. The genetically modified canine of claim 29, wherein the canine differs coat color, coat pattern, hair length, texture, hair growth/shedding, body size, leg length versus width, and skull shape, barking habit, allergen, and combination thereof than a canine in which the chromosomal region is not edited.

31. The genetically modified canine of claim 1, wherein the canine is an embryo, a juvenile, or an adult.

32. The genetically modified canine of claim 1, wherein the canine is a dog of a breed chosen from Labrador retriever, Golden retriever, Beagle, German shepherd, Dachshund, Yorkshire terrier, Boxer, Poodle, Shih tzu, Chihuahua, Miniature schnauzer, Pug dog, Pomeranian, Cocker spaniel, Rottweiler, Bulldog, Shetland sheepdog, Boston terrier, Miniature pinscher, Maltese, German shorthaired pointer, Doberman pinscher, Siberian husky, Pembroke welsh corgi, Basset hound, Bichon frise, and other existing or non-existing breeds.
